(19) United States
(12) Patent Application Publication
(10) Pub. No.: US 2011/0265810 A1
(43) Pub. Date: Nov. 3, 2011

(54) HAIR CARE COMPOSITIONS

(75) Inventors: PHILIP P. PELUSI, Pittsburgh, PA (US); FREDERICK H. BURMEISTER, New Hope, PA (US)

(73) Assignee: PHILIP PELUSI PHYTOLINK 4.0, INC., PITTSBURGH, PA (US)

(21) Appl. No.: 13/095,512

(22) Filed: Apr. 27, 2011

Related U.S. Application Data
(60) Provisional application No. 61/328,714, filed on Apr. 28, 2010.

(51) Int. Cl. A61K 8/92; A45D 7/06; A61Q 5/06; A61Q 5/00; A61K 8/72; A61K 8/81

(52) U.S. Cl. 132/206; 424/70.11; 424/70.15

(57) ABSTRACT

The present invention relates to a hair care composition which includes a volatile hydrocarbon-containing component; a non-volatile hydrocarbon-containing component; and an oil-soluble polymer component. The present invention also relates to a method for forming a hydrophobic barrier on hair to render the hair substantially resistant to moisture absorption which includes treating wet or damp hair with the hair care composition and applying heat to the treated hair. The hair care composition and method of the present invention is effective to reduce hair frizz and maintain a desired hair conformation.

### Field of the Invention

The present invention relates to hair care compositions including a volatile hydrocarbon-containing component, a non-volatile hydrocarbon-containing component, and an oil-soluble polymer component. These hair care compositions are particularly useful for application to hair to reduce frizz and to maintain a hair style.

### BACKGROUND OF THE INVENTION

A variety of hair care compositions are known in the art for varied uses. For example, there are hair cleansing compositions, hair conditioning compositions, and hair styling compositions. Many of these compositions which are known in the art are water-based formulations. Hair cleansing compositions are effective to remove soil from hair. The soil includes natural exudations from the scalp, environmental agents, and styling products. The soil can coat or deposit on the hair and scalp. Hair coated with such soil presents as being greasy in feel and appearance, heavy to the touch, possibly malodorous, and generally unable to maintain a desired style. Known cleansing compositions, typically include a combination of water and surface-active ingredients, such as soap or synthetic surfactants, and also may include a non-aqueous blend of starches. For example, dry shampoo or alcohol/hydrocarbon blends include a non-aqueous blend of starches that either adsorbs the soil or entrains it from the hair during evaporation. The combination of water and surface-active agents emulsifies the soil from the hair and scalp, allowing it to be rinsed away from the hair and scalp.

Cleansing compositions may also contain conditioning agents that deposit on the hair and scalp during rinsing with water. Such conditioning agents can include polymers, oils, waxes, protein hydrolysates, silicones, and mixtures and derivatives thereof. In addition, the conditioning composition can be a separate and different product from the cleansing composition.

Conditioning compositions that are known in the art are typically water-based formulations. However, there are also known conditioning compositions which include at least one of silicones; animal, mineral or vegetable oils; waxes; petrolatums; and greases. The water-based conditioning compositions typically include substituted cationic waxes, fatty alcohols, cationic polymers, hydrolyzed proteins and derivatives thereof, and fragrances. Such conditioning formulations impart comb-ability and manageability to the treated hair, thereby minimizing breakage during the styling process and resulting in shiny, healthy, and manageable hair. Conditioning compositions may also be effective to moisturize the hair by depositing water into the internal structure of the hair. Subsequent drying and styling processes can include air drying or heating by at least one of hot combs, blow dryers, and styling irons.

The use of heated instruments allows the potential for molecular rearrangement within the hair shaft. The keratinaceous structure of hair fibrils relies on bonding to create the characteristic structure of the hair. The bonds within the keratin fiber include cysteine bonds, salt bonds and hydrogen bonds. The salt and hydrogen bonds are influenced by the presence of water within the hair. These bonds are much weaker than the cysteine bonds and allow movement within the keratin structure to occur and allow for hair to frizz due to exposure to high humidity conditions. It is known that frizz can be temporarily abated by the use of hot implements, such as blow dryers, in combination with styling under tension, e.g., pulling with a brush or comb, or with tension styling in combination with flat or round heated irons. The presence of moisture and the influence of heat and tension allows the rearrangement of the weak salt and hydrogen bonds and creates a straight, although temporary, restyling of the hair. Unless some means of maintenance of the new style is present during the restyling process, reversion to the original configuration of the hair typically ensues.

The use of polymeric materials can prevent the immediate reversion from a straight conformation to hair's original configuration. There are known polymeric formulations, e.g., fixative polymers, which coat the surface of the hair with a cement- or glue-like layer that externally restricts the movement of the hair and reinforces the straight conformation. Polyvinyl pyrrolidone and derivatives thereof, or acrylic acid-based polymers and their derivatives thereof, have been widely used as fixative polymers. These polymeric compositions are formulated to resist humidity but do not provide a sufficiently hydrophobic barrier to water absorption to prevent the onset of frizz.

Hydrophobing agents, such as, silicones, for example, dimethicones and cyclomethicones, or fluorine-based polymers, are known to provide a hydrophobic barrier on the surface of the hair. Due to their molecular characteristics, hydrophobing agents also afford shine, lubricity, and water resistance to the hair. However, silicones and fluoropolymers are typically oily in nature and difficult to remove, which can contribute to buildup on the hair. In addition, silicones have been recognized for potential toxicity and negative impacts on the environment.

It is desired to provide improved hair care compositions that exhibit advantageous features, such as a hydrophobic barrier on hair, without exhibiting the disadvantages associated therewith, such as an oily buildup on the hair.

These needs and others are satisfied by the present invention.

### SUMMARY OF THE INVENTION

In one aspect, the present invention provides a hair care composition including a volatile hydrocarbon-containing component, a non-volatile hydrocarbon-containing component, and an oil-soluble polymer component. The hair care composition is effective to reduce hair fizz and maintain a desired hair conformation.

The volatile hydrocarbon-containing component can include at least one volatile hydrocarbon-containing compound selected from the group consisting of aliphatic hydrocarbon, straight chain hydrocarbon, branched chain hydrocarbon and mixtures thereof. The branched chain hydrocarbon can include an isoparaffin, such as but not limited to, a C₁₂-C₁₄ isoparaffin, such as but not limited to, isododecane.

The non-volatile hydrocarbon-containing component can include at least one non-volatile hydrocarbon-containing compound selected from the group consisting of aliphatic hydrocarbon, straight chain hydrocarbon, branched chain hydrocarbon, and mixtures thereof. The branched chain hydrocarbon can include an isoparaffin, such as but not limited to a C₁₃ to C₁₆ isoparaffin. The aliphatic hydrocarbon can include an alkane, such as but not limited to a C₁₃ to C₂₁ alkane or C₁₃ to C ₁₅ alkane, such as but not limited to isohexadecane.

The non-volatile hydrocarbon-containing component can include a volatile hydrocarbon-containing compound, such as but not limited to C₁₂ to C₁₄ isoparaffin.

The oil-soluble polymer component can be selected from co-polymer produced from a-olefin and vinyl pyrrolidone, terpolymer of vinyl caprolactam/vinyl pyrrolidone/dimethylaminoethyl/methacrylate, and mixtures thereof.

The volatile hydrocarbon-containing component can be present in an amount of from about 0.10 percent to about 50 percent by weight of the hair care composition and the non-volatile hydrocarbon-containing component can be present in an amount of from about 0.10 percent to about 20 percent by weight of the hair care composition. The hair care composition can further include an oil additive present in an amount of from about 0.10 percent to about 5.0 percent by weight of the hair care composition, and the remainder of the hair care composition can be alcohol.

In another aspect, the present invention can provide a method for forming a hydrophobic barrier on hair to render the hair substantially resistant to moisture absorption. The method includes treating at least partially wet hair with the hair care composition described above to produce treated hair and applying a source of heat to the treated hair to configure the treated hair in a desired hair style. In this method, the hair care composition can be a silicon-free composition and/or an anhydrous composition.

### DETAILED DESCRIPTION OF THE INVENTION

The present invention relates to a hair care composition, including a volatile hydrocarbon-containing component, a non-volatile hydrocarbon-containing component, and an oil-soluble polymer component. This hair care composition can be applied to hair to render the hair resistant to moisture absorption and resistant to fizz. Further, the composition aids in maintaining a desired style or conformation of the hair. The hair care composition is particularly useful in high humidity conditions which can cause hair to fizz.

Unless otherwise indicated, all numbers expressing quantities of ingredients, processing conditions and the like used in the specification and claims are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10.

The present disclosure describes several different features and aspects of the invention with reference to various exemplary non-limiting embodiments. It is understood, however, that the invention embraces numerous alternative embodiments, which may be accomplished by combining any of the different features, aspects, and embodiments described herein in any combination that one of ordinary skill in the art would find useful.

The volatile hydrocarbon-containing component can include at least one volatile hydrocarbon selected from a wide variety of such materials known in the art. Suitable examples for use in the present invention include aliphatic, straight chain and branched chain hydrocarbons and mixtures thereof. In an embodiment of the present invention, the branched hydrocarbon is highly branched, such as, C₁₂-C₁₄ isoparaffins. An example of an aliphatic, highly-branched hydrocarbon is isododecane which is a mixture of highly branched C₁₂ isoparaffins. Isododecane is commercially available under the trade name Permethyl™ Hydrocarbons (e.g., Permethyl 99A) from the Permethyl Corporation.

The non-volatile hydrocarbon-containing component can include at least one hydrocarbon selected from a wide variety of such materials known in the art. Suitable examples for use in the present invention include aliphatic, straight chain and branched chain hydrocarbons and mixtures thereof. In one embodiment, the non-volatile hydrocarbon-containing component includes a C₁₃-C₂₁ hydrocarbon. In a further embodiment, the non-volatile hydrocarbon-containing component includes a C₁₃-C₁₅ alkane, a C₁₃-C₁₆ isoparaffin, and mixtures thereof. The C₁₃-C₁₅ alkane can include aliphatic, straight chain and branched chain alkanes, and cycloalkanes. In yet a further embodiment, the non-volatile hydrocarbon-containing component includes isohexadecane.

In another embodiment of the present invention, the non-volatile hydrocarbon-containing component includes a silicone-free compound. The silicone-free compound can be capable to approximate the volatility and aesthetics of silicone-containing compound, such as cyclomethicone. In a further embodiment, the non-volatile hydrocarbon-containing component is a mixture of silicone-free compounds that replicates the physical properties of silicones which are commonly used in cosmetic applications. The non-silicone component can include at least one non-silicone component selected from a wide variety of materials known in the art that exhibit characteristics of silicon-components but are substantially silicone-free.

In yet another embodiment of the present invention, the non-volatile hydrocarbon-containing component can include a combination or mixture of non-volatile and volatile hydrocarbon-containing compounds. In one embodiment, the volatile hydrocarbon-containing compounds are present in an amount which is less than the amount of the non-volatile hydrocarbon-containing compounds.

In one embodiment, the non-volatile hydrocarbon-containing component includes a combination or mixture of a volatile compound, such as C₁₂-C₁₄ isoparaffin; and a non-volatile compound, such as C₁₃-C₁₆ isoparaffin, C₁₃ to C₂₁ alkane, and mixtures thereof. In a further embodiment, the volatile compound is present in an amount of about 20% to about 80% or about 30% to 35% by weight of the non-volatile hydrocarbon-containing component, and the non-volatile hydrocarbon-containing compound is present in an amount of about 20% to about 80% or about 60% to about 65% by weight of the non-volatile hydrocarbon-containing component. In a further embodiment, the non-volatile hydrocarbon-containing compound includes a C₁₃-C₁₆ isoparaffin which is present in an amount of about 20% to about 80% or about 50% to about 55% by weight of the non-volatile hydrocarbon-containing component and a C₁₃-C₂₁, or preferably a C₁₃-C₁₅, alkane which is present in an amount of about 5% to about 20% or about 10% by weight of the non-volatile hydrocarbon-containing component. An example of a non-silicone mixture of C₁₃-C₁₆ isoparaffin, C₁₂-C₁₄ isoparaffin and C₁₃ to C₁₅ alkane, is known under the trade name SiClone SR-5 and is commercially available from Presperse Incorporated.

The hair care composition of the present invention also includes an oil-soluble polymer component. The oil-soluble polymer component can be selected from a wide variety of such materials known in the art, such as but not limited to, combinations or mixtures of co-polymers produced from a-olefins and vinyl pyrrolidone. In one embodiment, the oil-soluble polymeric component includes a mixture of alkylated polyvinyl pyrrolidone and hexadecene in a 20/80 ratio. In another embodiment, the oil-soluble polymeric component includes a terpolymer of vinyl caprolactam/vinyl pyrrolidone/dimethylaminoethyl/methacrylate. The terpolymer can be in the form of a solution, e.g., by mixing with a solvent, such as but not limited to, ethanol. Non-limiting examples of commercially available oil-soluble polymeric materials suitable for use in the present invention include Antaron-Ganex® V-216 and Gaffix® (both from ISP Corporation).

The volatile hydrocarbon-containing component, the non-volatile hydrocarbon-containing component, and the oil-soluble polymer component are combined to form the hair care composition of the present invention. The hair care composition can further include other optional ingredients that are known for use in such compositions, such as alcohol, fragrances, oils, solvents and combinations thereof.

In one embodiment, the volatile hydrocarbon-containing component is isododecane, the non-volatile hydrocarbon-containing component is a combination of C₁₃-C₁₆ isoparaffin, C₁₂-C₁₄ isoparaffin and C₁₃-C₁₅ alkane, and the oil soluble polymer component is a mixture of alkylated polyvinyl pyrrolidone and hexadecene.

The amounts of the components and compounds used in preparing the hair care composition of the present invention can vary. In one embodiment, the volatile hydrocarbon-containing component is present in an amount of from about 0.10 percent to about 50 percent; the non-volatile hydrocarbon-containing component is present in an amount of from about 0.10 percent to about 20 percent; and the oil-soluble polymer component is present in an amount of from about 0.10 percent to about 10 percent. In further embodiment, the hair care composition also includes an oil additive present in an amount of from about 0.10 percent to about 5.0 percent and the remainder of the composition is alcohol.

In one embodiment of the present invention, the volatile hydrocarbon-containing component is isododecane, e.g., Permethyl™ 99A, the non-volatile hydrocarbon-containing component is a non-silicone component including a mixture of C₁₃-C₁₆ isoparaffin, C₁₂-C₁₄ isoparaffin, and C₁₃-C₁₅ alkane, e.g., SiClone SR-5, the oil-soluble polymeric material is a includes co-polymers produced from a-olefins and vinyl pyrrolidone, e.g., Antaron-Ganex® V-216 and Gaffix® (both from ISP Corporation), the oil includes but is not limited to isopropyl myristate, isopropyl palmitate, mineral oil, isononyl isononanoate, polybutenes, polyisobutenes and hydrogenated polyisobutenes, such as lavender essential oil, and the alcohol is ethyl alcohol, such as, Alcohol SDA-40 (200 proof).

In one embodiment of the present invention, the hair care composition is applied to damp or wet hair to be treated, and a source of heat is subsequently applied thereto. The source of heat can include a wide variety of heated styling tools that are known in the art, such as, but not limited to, hair dryers, flat irons and the like. Not intending to be bound by any particular theory, it is believed that application of heat to damp or wet hair facilitates the evaporation of the volatile component and produces steam that with the application of tension during styling of the hair alters and rearranges certain of the internal bonds within the hair shaft to reduce frizz and create an enhanced straightened conformation. Thus, the hair care of composition of the present invention is applied to the damp or wet hair, and the subsequent application of a heat treatment results in forming a hydrophobic barrier such that the hair is resistant to moisture absorption. Further, without intending to be bound by any particular theory, it is believed that the application of heat (e.g., during the hair styling process) to the hair care composition of the present invention results in substantial evaporation of the hydrophobing composition (e.g., the volatile and non-volatile components, the polymer component and the alcohol) and therefore, little (e.g., minimal) residual remains on the hair, thereby minimizing or eliminating the problem of product build-up on the hair.

The present invention does not require the presence of many of the compounds that are included in known hair care compositions in order to produce a hydrophobic barrier, such as, but not limited to, silicone-containing compounds, such as dimethicones and cyclomethicones, and waxes. In one embodiment, the hair care composition of the present invention is a non-silicone based formulation or silicone-free. Further, known hair care compositions may include water-based formulations. In the present invention, the hair care composition can include substantially less water as compared to known compositions or the hair care composition of the present invention can be essentially water-free. In one embodiment, the present invention relates to an anhydrous hair care composition.

In another embodiment of the present invention, two non-silicone-containing hydrophobic components, such as isoparaffin and alkane, are combined with a polymeric component, such as Antaron-Ganex® V-216 and Gaffix® (both from ISP Corporation), to produce the hair care composition of the present invention.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting to the scope of the invention.

What is claimed is:

1. A hair care composition, comprising:
a volatile hydrocarbon-containing component;
a non-volatile hydrocarbon-containing component; and
an oil-soluble polymer component,
wherein, the hair care composition is effective to reduce hair frizz and maintain a desired hair conformation.

2. The hair care composition of claim 1, wherein the volatile hydrocarbon-containing component comprises at least one volatile hydrocarbon-containing compound selected from the group consisting of aliphatic hydrocarbon, straight chain hydrocarbon, branched chain hydrocarbon, and mixtures thereof.

3. The hair care composition of claim 2, wherein the branched chain hydrocarbon comprises an isoparaffin.

4. The hair care composition of claim 3, wherein the isoparaffin comprises a C₁₂-C₁₄ isoparaffin.

5. The hair care composition of claim 4, wherein the C₁₂-C₁₄ isoparaffin comprises isododecane.

6. The hair care composition of claim 1, wherein the non-volatile hydrocarbon-containing component comprises at least one non-volatile hydrocarbon-containing compound selected from the group consisting of aliphatic hydrocarbon, straight chain hydrocarbon, branched chain hydrocarbon, and mixtures thereof.

7. The hair care composition of claim 6, wherein the branched chain hydrocarbon comprises an isoparaffin.

8. The hair care composition of claim 7, wherein the isoparaffin comprises a C₁₃-C₁₆ isoparaffin.

9. The hair care composition of claim 6, wherein the aliphatic hydrocarbon comprises an alkane.

10. The hair care composition of claim 9, wherein the alkane comprises a C₁₃-C₂₁ alkane.

11. The hair care composition of claim 10, wherein the alkane comprises a C₁₃ to C₁₅ alkane.

12. The hair care composition of claim 11, wherein the C₁₃ to C₁₅ alkane comprises isohexadecane.

13. The hair care composition of claim 1, wherein the non-volatile hydrocarbon-containing component comprises a volatile hydrocarbon-containing compound.

14. The hair care composition of claim 13, wherein the volatile hydrocarbon-containing compound is C₁₂ to C₁₄ isoparaffin.

15. The hair care composition of claim 1, wherein the oil-soluble polymer component is selected from the group consisting of co-polymer produced from a-olefin and vinyl pyrrolidone, terpolymer of vinyl caprolactam/vinyl pyrrolidone/dimethylaminoethyl/methacrylate, and mixtures thereof.

16. The hair care composition of claim 1, wherein the volatile hydrocarbon-containing component is present in an amount of from about 0.10 percent to about 50 percent by weight of the hair care composition and the non-volatile hydrocarbon-containing component is present in an amount of from about 0.10 percent to about 20 percent by weight of the hair care composition.

17. The hair care composition of claim 16, further comprising an oil additive present in an amount of from about 0.10 percent to about 5.0 percent by weight of the hair care composition, and the remainder of the hair care composition being alcohol.

18. A method for forming a hydrophobic barrier on hair to render the hair substantially resistant to moisture absorption, comprising:
treating at least partially wet hair with a hair care composition which comprises:
a volatile hydrocarbon-containing component;
a non-volatile hydrocarbon-containing component; and
an oil-soluble polymer component, to produce treated hair;
applying a source of heat to the treated hair to configure the treated hair in a desired hair style.

19. The method of claim 18, wherein the hair care composition comprises a silicone-free composition.

20. The method of claim 18, wherein the hair care composition comprises an anhydrous composition.
